(19) United States
(12) Patent Application Publication
(10) Pub. No.: US 2017/0360289 A1
(43) Pub. Date: Dec. 21, 2017

(54) EXAMINATION TOOL

(71) Applicant: Coden Co., Ltd., Tokyo (JP)

(72) Inventors: Yoshiharu Koda, Tokyo (JP); Kojiro KODA, Tokyo (JP)

(21) Appl. No.: 15/627,889

(22) Filed: Jun. 20, 2017

(30) Foreign Application Priority Data
Jun. 21, 2016 (JP) 2016122823

(51) Int. Cl. A61B 1/227; A61B 1/00; A61B 5/00; A61B 1/06

(52) U.S. Cl. CPC A61B 1/227; A61B 1/00048; A61B 1/00052; A61B 5/0033; A61B 1/06

(57) ABSTRACT

An imaging tube 210 includes an imaging lens and an illumination unit. A cap part 230 including a penetration part in which the imaging tube 210 penetrates is included at a leading end part of amain body part 220. A protective tube 212 that protects the entire circumference of the imaging tube 210 is attached to the imaging tube 210. A holding part 235 that holds an end part of the protective tube 212 with the imaging tube 210 is formed in the penetration part of the cap part 230. When being rotated in a first direction, the cap part 230 is moved in a direction opposite to a protruding direction of the imaging tube 210. When being rotated in a second direction opposite to the first direction, the cap part 230 is moved in the protruding direction of the imaging tube 210.

### CROSS-REFERENCE TO RELATED APPLICATION

The present application claims priority under 35 U.S.C. §119 to Japanese Patent Application No. 2016-122823, filed Jun. 21, 2016. The contents of this application are incorporated herein by reference in their entirety.

### BACKGROUND

### 1. Technical Field

The present invention relates to an examination tool to observe an inner part of an ear such as an ear canal and an eardrum.

### 2. Description of Related Arts

Maintenance of an inner part of an ear is necessary not only for a human but also for a small/medium animal. Ear canal cleaning of a human is routinely performed with various cleaning methods. A structure of an ear canal of a human is horizontal. A structure in a straight line is normal. Some people have structures curved in some degree. On the other hand, a structure of an ear canal of a small/medium animal is in a curved line toward a lower inner surface. An eardrum is at a leading end and an ear canal is narrow. In an inner part of an ear, earwax in various conditions such as dry earwax, wet earwax, and dandruff-type earwax is generated. Specifically, an inner part of an ear in a small/medium animal is likely to be parasitized by an animalcule such as a tick and louse. Thus, an ear canal, an eardrum, and a part near the eardrum are likely to be contaminated. A disease of the eardrum is caused very frequently. Thus, in order to detect abnormality in an inner part of an ear early in a human and a small/medium animal, it is desirable that the inner part of the ear is examined regularly and that the inner part of the ear is taken care of.

In Japanese Patent Application Laid-Open No. 2014-138858, an otoscope with which it is possible to diagnose inflammation or infection in an inner part of an ear by imaging of a three-dimensional image or a spectrum image in an inner part of an earhole is disclosed.

### SUMMARY

The otoscope disclosed in Japanese Patent Application Laid-Open No. 2014-138858 can improve a diagnosis of inflammation or infection in an inner part of an ear. However, a device itself as the otoscope is large-scaled and cannot be easily used by an ear, nose and throat hospital or an animal hospital. Also, since there is no low-cost examination tool, with which a part near an ear canal or an eardrum can be easily examined, for treatment of an eardrum of a small/medium animal, a high cost is necessary to introduce the above otoscope. Thus, it is difficult for a small-scale ear, nose and throat hospital or animal hospital to purchase the above otoscope.

Many hospitals have a desire to introduce such otoscope. However, there are many hospitals hesitating to introduce the otoscope since the otoscope is a large-scale device and requires a high introduction cost.

Also, many animal hospitals have a desire to observe an inner part of an ear with a clear image. However, even with the otoscope disclosed in Japanese Patent Application Laid-Open No. 2014-138858, it is difficult to observe an inner part of an ear of an animal, humidity in an ear canal of which is higher than that of a human, without fogging of a lens of a camera.

Furthermore, in order to prevent infection, the otoscope usually requires work to disinfect a part inserted into the ear canal after use. For this reason, it cannot be said that working efficiency is good, and there is a concern about reliability in terms of hygiene.

The present invention is provided to respond to such various conventional demands and is to provide an examination tool with high reliability in terms of hygiene which tool is simple in structure and inexpensive to introduce and with which tool it is possible to observe an inner part of an ear with a clear image.

An examination tool according to an aspect of the present invention for achieving the above object is an examination tool including: an imaging tube that is inserted into an ear canal to image an inner part of an ear; and a main body part from a leading end part of which the imaging tube protrudes, wherein the imaging tube includes, at a leading end part thereof, an imaging lens that images the inner part of the ear and an illumination unit that is provided adjacent to the imaging lens and that illuminates the inner part of the ear, a cap part including a penetration part in which the imaging tube penetrates is included at the leading end part of the main body part, a protective tube that protects an entire circumference of the imaging tube is attached to the imaging tube, a holding part that holds an end part of the protective tube with the imaging tube is formed in the penetration part of the cap part, and the cap part is moved in a direction opposite to a protruding direction of the imaging tube and is fixed to the leading end part when being rotated in a first direction along a circumferential direction of the main body part, and is moved in the protruding direction of the imaging tube, is detached from the leading end part, and moves the protective tube held by the holding part in the protruding direction of the imaging tube when being rotated in a second direction opposite to the first direction.

An examination tool according to another aspect of the present invention for achieving the above object is an examination tool including: an imaging tube that is inserted into an ear canal to image an inner part of an ear; and a main body part from a leading end part of which the imaging tube protrudes, wherein the imaging tube includes, at a leading end part thereof, an imaging lens that images the inner part of the ear and an illumination unit that is provided adjacent to the imaging lens and that illuminates the inner part of the ear, a cap part including a protective part in which the imaging tube penetrates and which protects an entire circumference of the imaging tube is included at the leading end part of the main body part, and the cap part is moved in a direction opposite to a protruding direction of the imaging tube and is fixed to the leading end part when being rotated in a first direction along a circumferential direction of the main body part, and is moved in the protruding direction of the imaging tube and detached from the leading end part when being rotated in a second direction opposite to the first direction.

According to the examination tool according to the present invention, an introduction cost becomes low with a simple structure, an inner part of an ear can be observed with a clear image, and high reliability can be secured in terms of hygiene.

### BRIEF DESCRIPTION OF THE DRAWINGS

FIG. 1 is a configuration diagram of an ear canal examination device including an examination tool according to a first embodiment;

FIG. 2 is a side view of an imaging tube and a main body part of the examination tool according to the first embodiment;

FIG. 3A is a view illustrating a partly-broken side surface of the examination tool according to the first embodiment;

FIG. 3B is a cross section of a cap part of the examination tool according to the first embodiment;

FIG. 4 is a perspective view of the examination tool according to the first embodiment;

FIG. 5A is a view illustrating the examination tool according to the first embodiment in a perspective manner;

FIG. 5B is a cross section of a penetration part of the cap part according to the first embodiment;

FIG. 6 is a cross-sectional view of the main body part and the imaging tube of the examination tool according to the first embodiment;

FIG. 7 is a view for describing operation of the examination tool according to the first embodiment;

FIG. 8 is a view for describing the operation of the examination tool according to the first embodiment;

FIG. 9 is a view for describing the operation of the examination tool according to the first embodiment;

FIG. 10 is a view for describing the operation of the examination tool according to the first embodiment;

FIG. 11 is a view for describing the operation of the examination tool according to the first embodiment;

FIG. 12 is a view for describing the operation of the examination tool according to the first embodiment;

FIG. 13 is a configuration diagram of an examination tool according to a second embodiment;

FIG. 14A is a view illustrating a partly-broken side surface of the examination tool according to the second embodiment;

FIG. 14B is a cross section of a cap part of the examination tool according to the second embodiment;

FIG. 15 is a perspective view of the examination tool according to the second embodiment;

FIG. 16 is a view for describing operation of the examination tool according to the second embodiment;

FIG. 17 is a view for describing the operation of the examination tool according to the second embodiment; and

FIG. 18 is a view for describing the operation of the examination tool according to the second embodiment;

FIG. 19A is a cross section of a cap part of the examination tool according to the third embodiment;

FIG. 19B is a Left side view of a cap part of the examination tool according to the third embodiment;

FIG. 19C is a Right side view of a cap part of the examination tool according to the third embodiment;

FIG. 20 is a configuration diagram of an examination tool according to third embodiment.

### DETAILED DESCRIPTION

In the following, a [first embodiment] and a [second embodiment] of an examination tool according to the present invention will be described in detail.

### First Embodiment

The first embodiment is an examination tool with which it is possible to observe an inner part of an ear such as an ear canal and an eardrum with a clear image and in which a protective tube can be replaced after observation of the inner part of the ear.

### Configuration of Ear Canal Examination Device

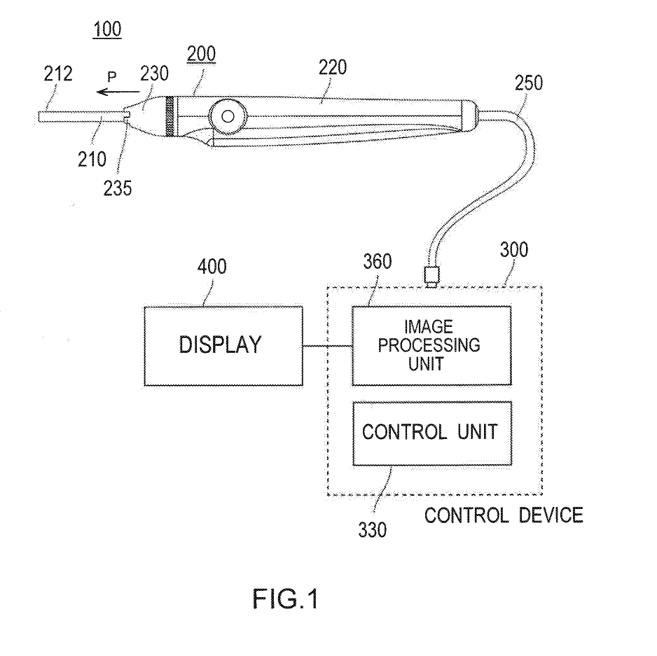

FIG. 1 is a configuration diagram of an ear canal examination device including an examination tool according to the first embodiment. As illustrated in FIG. 1, an ear canal examination device 100 includes an examination tool 200, a control device 300, and a display 400.

With the examination tool 200, it is possible to examine (observe) an inner part of an ear of a human or an animal. The examination tool 200 includes an imaging tube 210 that is inserted into the ear canal to image an inner part of an ear, and a main body part 220 from a leading end part of which the imaging tube 210 protrudes.

Although not illustrated in FIG. 1, the imaging tube 210 includes an imaging lens, which images an inner part of an ear, at a leading end part on a side inserted into an ear canal and an illumination unit that illuminates the inner part of the ear. The illumination unit is provided adjacent to the imaging lens at the leading end. This is to warm the imaging lens with residual heat of the illumination unit and to prevent fogging of the imaging lens. A detachable cylindrical protective tube 212 is attached to the imaging tube 210. The protective tube 212 protects the entire circumference of the imaging tube 210. The length of the protective tube 212 is longer than the length of a protruding part of the imaging tube 210. This is to prevent the imaging lens and the illumination unit from contacting the ear canal. Also, it is preferable that the protective tube 212 is formed of a flexible and soft material so as not to damage an inner wall of the ear canal.

To the leading end part of the main body part 220 from which end part the imaging tube 210 protrudes, a cap part 230 that can be attached to/detached from the leading end part by a rotation is attached. The cap part 230 includes a penetration part where the imaging tube 210 penetrates. A holding part 235 that holds an end part of the protective tube 212 with the imaging tube 210 is formed in the penetration part. When the protective tube 212 is slid from a leading end direction of the imaging tube 210 to cover the imaging tube 210 and a leading end of the protective tube 212 is inserted into the holding part 235, the protective tube 212 is held by the holding part 235.

When being rotated in a first direction along a circumferential direction of the main body part 220 (such as rightward direction), the cap part 230 is moved in a direction opposite to a protruding direction of the imaging tube 210 (direction of arrow P) and is fixed to the leading end part of the main body part 220. When being rotated in a second direction (such as leftward direction) opposite to the first direction, the cap part 230 is moved in the protruding direction of the imaging tube 210 (direction of arrow P), is detached from the leading end part of the main body part 220, and moves the protective tube 212 held by the holding part in the protruding direction of the imaging tube.

A camera (not illustrated) is built in the main body part 220. The camera and the imaging lens are connected to each other by an optical fiber. Thus, an image of an inner part of an ear which image is captured by the imaging lens is transmitted to the camera via the optical fiber. The camera is connected to the control device 300 via a cable 250 drawn out from a rear end part of the main body part 220. Note that the camera is provided in the main body part 220 in the first embodiment. However, the camera may be directly attached to the imaging lens. In that case, an external dimension of the camera is about 2 to 4 mm.

The control device 300 includes a control unit 330 and an image processing unit 360. The control unit 330 totally controls operations of the camera and the illumination unit included in the main body part 220, and the image processing unit 360. The image processing unit 360 processes an image transmitted from the camera and displays the image on the display 400.

### [Operation of Ear Canal Examination Device]

When the examination tool 200 according to the first embodiment is used, the cap part 230 is attached to the leading end part of the main body part 220, the protective tube 212 is put on the imaging tube 210, and the protective tube 212 is held by the holding part 235 of the cap part 230. The leading end part of the imaging tube 210 is inserted into an ear canal in this state, and a state of an inner part of an ear is observed with reference to an image displayed on the display 400.

When the observation of the state of the inner part of the ear is over, the cap part 230 is detached from the leading end part of the main body part 220, the protective tube 212 moved in the protruding direction of the imaging tube 210 is detached from the imaging tube 210, and the cap part 230 is detached from the main body part 220.

As described above, a structure of the examination tool 200 according to the first embodiment is simple. Also, inexpensive general-purpose products can be used as the control device 300 and the display 400. Thus, the examination tool 200 and the ear canal examination device 100 according to the first embodiment have simple structures and an introduction cost thereof becomes low. In addition, since fogging of the imaging lens can be prevented with heat of the illumination unit, the inner part of the ear can be observed with a clear image. Furthermore, since the protective tube 212 can be replaced each time being used, high reliability can be secured in terms of hygiene. [Configuration of Examination Tool]

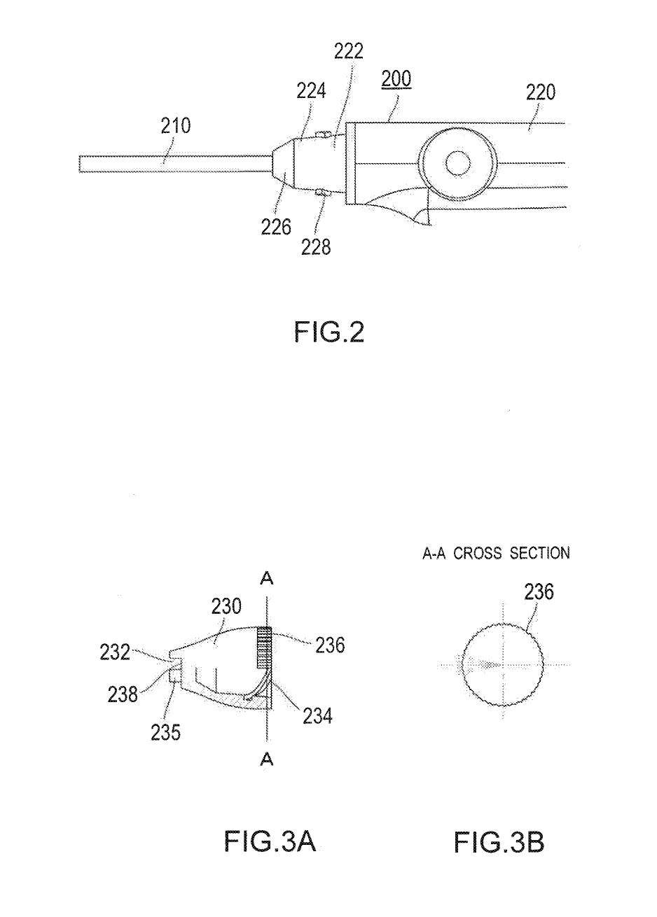

Next, with reference to FIGS. 2 to 6, the configuration of the examination tool 200 according to the first embodiment will be described in detail.

FIG. 2 is a side view of the imaging tube 210 and the main body part 220 of the examination tool 200 according to the first embodiment.

The imaging tube 210 protrudes from the leading end part 222 of the main body part 220. The leading end part 222 includes a first tapered part 224 having a gentle taper angle, and a second tapered part 226 having a taper angle steeper than that of the first tapered part. In the first tapered part 224, locking pieces 228 are provided at constant angular intervals in the circumferential direction of the leading end part 222.

FIG. 3A is a view illustrating a partly-broken side surface of the examination tool 200 according to the first embodiment. FIG. 3B is a cross section of the cap part 230 of the examination tool 200 according to the first embodiment.

The cap part 230 includes a bowl-shaped hollow shape formed along a shape of the leading end part 222 illustrated in FIG. 2. In the cap part 230, a penetration part 232 where the imaging tube 210 illustrated in FIG. 2 penetrates is formed. In the penetration part 232, the holding part 235 that holds the protective tube 212 (see FIG. 1) with the imaging tube 210 is formed.

The holding part 235 is a protrusion that protrudes in the protruding direction of the imaging tube 210 from the penetration part 232 of the cap part 230. The diameter of the penetration part 232 is equal to the diameter of the imaging tube 210 or larger than the diameter of the imaging tube 210. The penetration part 232 includes an abutment part 238 to which an end part of the protective tube 212 illustrated in FIG. 1 is abutted. The distance from a surface of the holding part 235 to a surface of the imaging tube 210 is equal to the thickness of the protective tube 212 or shorter than the thickness of the protective tube 212.

Engagement grooves 234 that engage with the locking pieces 228 illustrated in FIG. 2 are included at constant angular intervals in the circumferential direction in the inner peripheral part of the cap part 230.

Thus, when the cap part 230 is abutted to the leading end part 222 illustrated in FIG. 2 and the cap part 230 is rotated in the first direction (such as rightward direction), the locking pieces 228 are engaged with the engagement grooves 234 and the cap part 230 is moved in the direction opposite to the protruding direction of the imaging tube 210 (rightward direction in FIG. 2) and is firmly fixed to the leading end part 222 of the main body part 220.

On the other hand, when the cap part 230 is rotated in the second direction (such as leftward direction) opposite to the first direction, the cap part 230 is moved in the protruding direction of the imaging tube 210 (leftward direction in FIG. 2) until the engagement grooves 234 are detached from the locking pieces 228.

Anti-slip grooves 236 are formed in an end part with the largest diameter of the cap part 230. The anti-slip grooves 236 are provided in the entire circumference of the cap part 230 as illustrated in an A-A cross section.

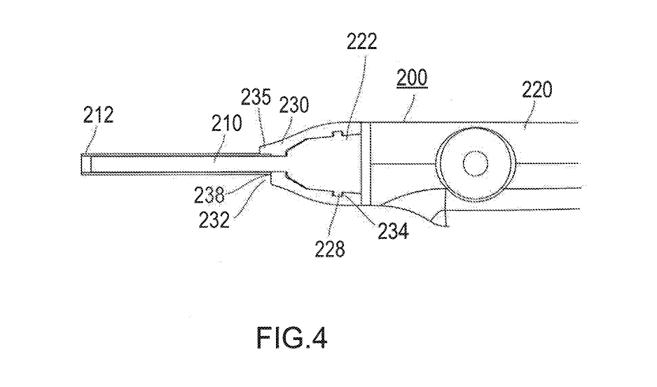

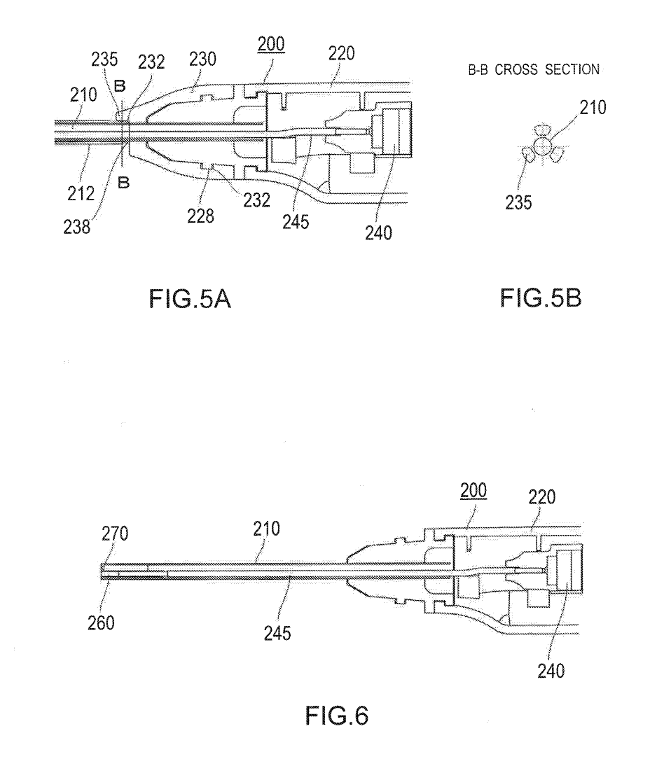

FIG. 4 is a perspective view of the examination tool 200 according to the first embodiment. FIG. 5A is a view illustrating the examination tool 200 according to the first embodiment in a perspective manner. FIG. 5B is a cross section of a penetration part of the cap part 230 according to the first embodiment. FIG. 6 is a cross-sectional view of the main body part 220 and the imaging tube 210 of the examination tool 200 according to the first embodiment.

The cap part 230 is attached to the leading end part 222 of the main body part 220. The cap part 230 is firmly attached to the main body part 220 by the locking pieces 228 provided at the leading end part 222 and the engagement grooves 234 in the cap part 230 itself. The imaging tube 210 protruding from the main body part 220 penetrates the penetration part 232 of the cap part 230. As illustrated in a B-B cross section, three holding parts 235 provided with angular differences of 120 degrees in the circumferential direction are provided in the penetration part 232. The protective tube 212 protects the entire circumference of the imaging tube 210 from the abutment part 238 of the penetration part 232 in the cap part 230 to the leading end part of the imaging tube 210.

As illustrated in FIG. 5A, 5B, the protective tube 212 is held by the holding parts 235 with an end part thereof being abutted to the abutment part 238 and being inserted into a space between the imaging tube 210 and the holding parts 235. The protective tube 212 is longer than the imaging tube 210. As illustrated in FIG. 4, the protective tube 212 protects the entire circumference of the imaging tube 210 in a state of protruding more than the leading end part of the imaging tube 210.

When the cap part 230 is rotated in the second direction (such as leftward direction) opposite to the first direction (such as rightward direction), the cap part 230 and the protective tube 212 are moved in the protruding direction of the imaging tube 210 (leftward direction in drawing).

Thus, the end part of the protective tube 212 is pushed by the abutment part 238, the leading end part of the protective tube 212 is greatly deviated from the leading end part of the imaging tube 210, and it becomes easier to pinch the leading end part of the protective tube 212. Thus, replacement of the protective tube 212 becomes easy.

As illustrated in FIGS. 5 and 6, a camera 240 to image an inner part of an ear is built in the main body part 220. An optical fiber 245 is attached to the camera 240. The optical fiber 245 is connected, through the imaging tube 210, to an imaging lens 260 provided at the leading end part of the imaging tube 210. Thus, the camera 240 is connected to the imaging lens 260 via the optical fiber 245. Also, the imaging lens 260 and an LED 270, which functions as the illumination unit, are arranged side by side in a radial direction in the leading end part of the imaging tube 210.

Thus, the imaging lens 260 is prevented, with the heat generated by the LED 270, from being fogged due to the humidity inside an ear canal. Note that the inner part of the ear is irradiated only with the LED 270 in the first embodiment. However, an LED may be arranged along an outer periphery of the leading end part of the imaging tube 210 in addition to the LED 270.

### [Operation of Examination Tool]

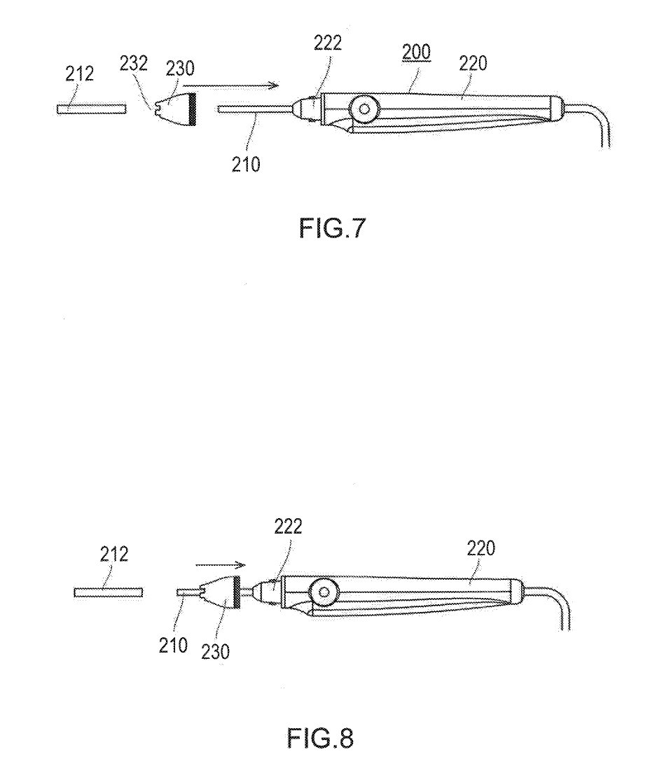

FIGS. 7 to 12 are views for describing the operation of the examination tool 200 according to the first embodiment. As illustrated in FIGS. 7 and 8, when the examination tool 200 is used, first, the imaging tube 210 is inserted into the penetration part 232 of the cap part 230, and the cap part 230 is slid along the imaging tube 210 in a direction of an illustrated arrow until the cap part 230 is abutted to the leading end part 222.

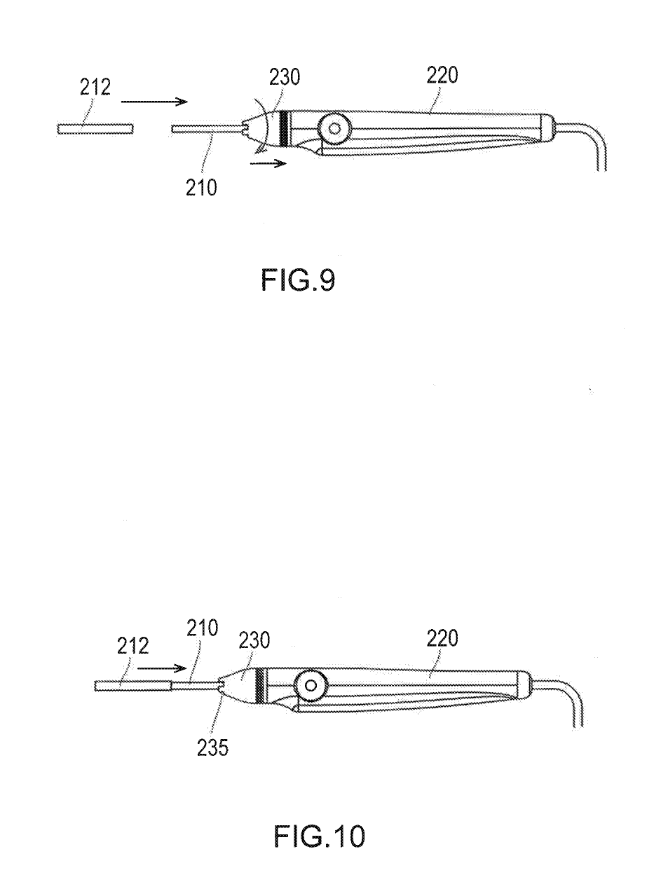

When the cap part 230 is abutted to the leading end part 222, the cap part 230 is rotated in the first direction (rightward direction in drawing) as illustrated in FIG. 9. Then, the engagement grooves 234 illustrated in FIG. 3A, 3B are engaged with the locking pieces 228 in FIG. 2 and the cap part 230 is further moved in the direction of the illustrated arrow and is firmly attached to the main body part 220. In this state, as illustrated in FIG. 10, the protective tube 212 is moved in the direction of the illustrated arrow until being abutted to the abutment part 238 illustrated in FIG. 5A, 5B in such a manner that the end part of the protective tube 212 is held by the holding parts 235.

In this state, since the imaging tube 210 is covered and protected by the protective tube 212, the imaging tube 210 is inserted into the ear canal and a state of the inner part of the ear is observed. During the observation of the state of the inner part of the ear, the LED 270 illustrated in FIG. 6 is on and the state of the inner part of the ear is imaged by the camera 240. Although humidity inside the ear canal is high depending on a kind of an animal, the imaging lens 260 is not fogged since the imaging lens 260 is warmed by the heat of the LED 270.

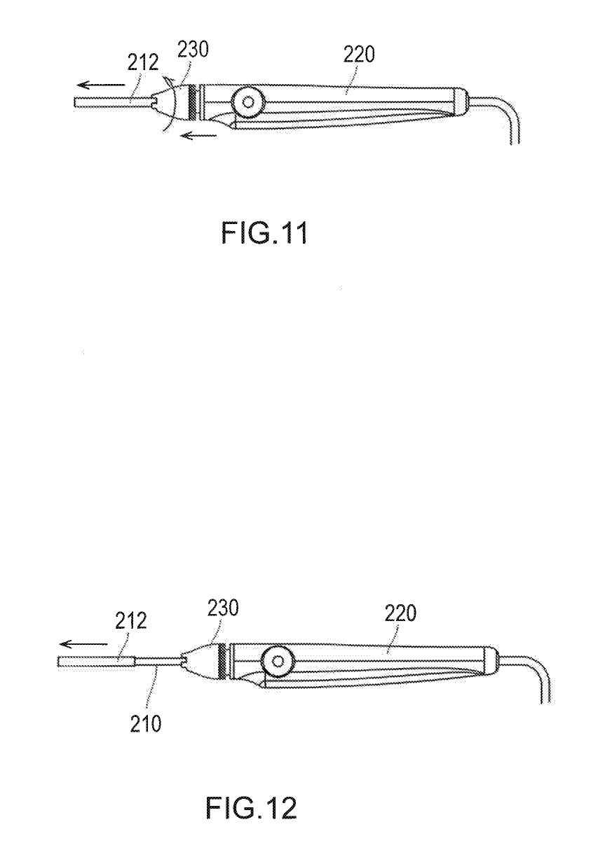

When the observation of the state of the inner part of the ear is over, the cap part 230 is rotated in the second direction (leftward direction in drawing) as illustrated in FIG. 11. Then, the engagement grooves 234 illustrated in FIG. 3A, 3B are detached from the locking pieces 228 in FIG. 2 and the cap part 230 is moved in a direction of an illustrated arrow. Thus, the protective tube 212 is moved in the direction of the illustrated arrow as the abutment part 238 of the cap part 230 illustrated in FIG. 5A, 5B is moved, and it becomes easier to detach the protective tube 212 from the imaging tube 210.

By an operator, the protective tube 212 that becomes easier to be detached is moved in the direction of the illustrated arrow with an end thereof being pinched and is detached from the imaging tube 210 as illustrated in FIG. 12.

As described above, according to the examination tool of the first embodiment, an introduction cost becomes low since a structure thereof is simple compared to a conventional one. Since fogging of the imaging lens 260 is designed to be prevented, it is possible to observe an inner part of an ear with a clear image. Moreover, since the protective tube 212 can be replaced, high reliability can be secured in terms of hygiene.

### Second Embodiment

In the second embodiment, a part corresponding to the protective tube 212 of the first embodiment is integrated with the cap part 230. With an examination tool of the second embodiment, it is also possible to observe an inner part of an ear with a clear image. Also, it is possible to replace a protective tube after observation of the inner part of the ear.

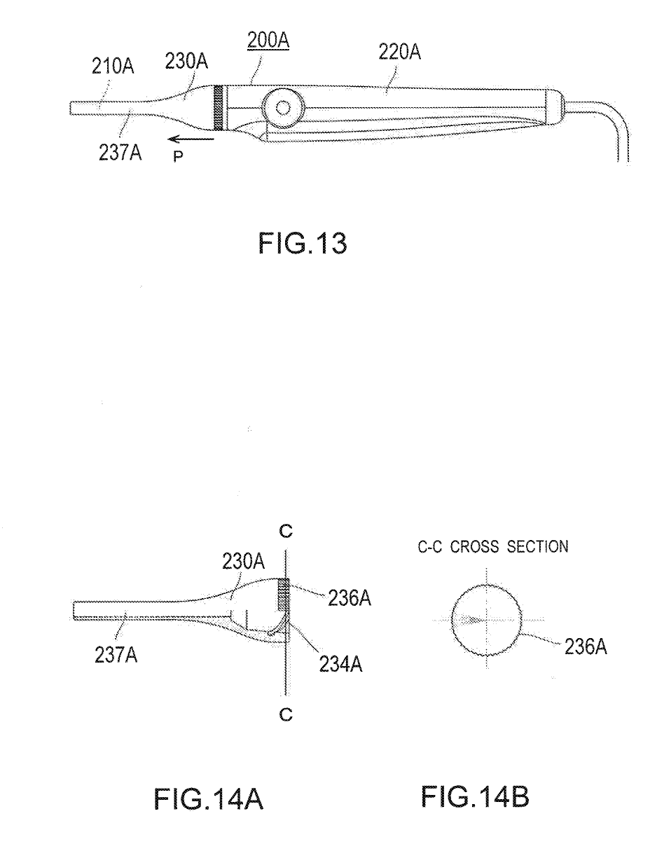

FIG. 13 is a configuration diagram of the examination tool according to the second embodiment An examination tool 200A of the second embodiment is different from the examination tool 200 of the first embodiment only in a structure of a cap part 230A. Configurations of the other parts are the same as those of the first embodiment.

As illustrated in FIG. 13, the examination tool 200A includes an imaging tube 210A that is inserted into an ear canal to image an inner part of an ear and a main body part 220A from a leading end part of which an imaging tube 210A protrudes.

Although not illustrated in FIG. 13, the imaging tube 210A includes an imaging lens, which images an inner part of an ear, at a leading end part on a side inserted into an ear canal and an illumination unit that illuminates the inner part of the ear. The illumination unit is provided adjacent to the imaging lens. This is to warm the imaging lens with heat of the illumination unit and to prevent fogging of the imaging lens. A cap part 230A including a protective part 237A in which the imaging tube 210A penetrates and which protects the entire circumference of the imaging tube 210A is included at the leading end part of the main body part. 220A. The protective part 237A is formed integrally with the cap part 230A.

The protective part 237A of the cap part 230A protects the entire circumference of the imaging tube 210A in a state of protruding more than the leading end part of the imaging tube 210A. This is to prevent the imaging lens and the illumination unit provided at the leading end part of the imaging tube 210A from contacting an ear canal.

When being rotated in a first direction (such as rightward direction) along a circumferential direction of the main body part 220A, the cap part 230A is moved in a direction opposite to a protruding direction of the imaging tube 210A (direction of arrow P) and is fixed to the leading end part of the main body part 220A. When being rotated in a second direction (such as leftward direction) opposite to the first direction, the cap part 230A is moved in the protruding direction of the imaging tube 210A (direction of arrow P) and is detached from the leading end part of the main body part 220A.

A camera (not illustrated) is built in the main body part 220A. The camera and the imaging lens are connected to each other by an optical fiber. Thus an image of an inner part of an ear which image is captured by the imaging lens is transmitted to the camera via the optical fiber.

FIG. 14A is a view illustrating a partly-broken side surface of the examination tool 200A according to the second. embodiment. FIG. 14B is a a cross section of the cap part 230A of the examination tool 200A according to the second embodiment.

The cap part 230A has a bowl-like hollow shape formed along a shape of the leading end part 222 illustrated in FIG. 2. The cap part 230A includes a protective part 237A in which the imaging tube 210 illustrated in FIG. 2 penetrates and which protects the entire circumference of the imaging tube 210A.

Engagement grooves 234A that engage with the locking pieces 228 illustrated in FIG. 2 are included at constant angular intervals in a circumferential direction in an inner peripheral part of the cap part 230A.

Thus, when the cap part 230A is abutted to the leading end part 222 illustrated in FIG. 2 and the cap part 230A is rotated in the first direction (such as rightward direction), the locking pieces 228 are engaged with the engagement grooves 234A and the cap part 230A is moved in the direction opposite to the protruding direction of the imaging tube 210A and is firmly fixed to the leading end part of the main body part 220A.

On the other hand, when the cap part 230A is rotated in the second direction (such as leftward direction) opposite to the first direction, the cap part 230A is moved in the protruding direction of the imaging tube 210A until the engagement grooves 234A are detached from the locking pieces 228.

Anti-slip grooves 236A are formed in an end part with the largest diameter of the cap part 230A. The anti-slip grooves 236A are provided in the entire circumference of the cap part 230A as illustrated in a C-C cross section.

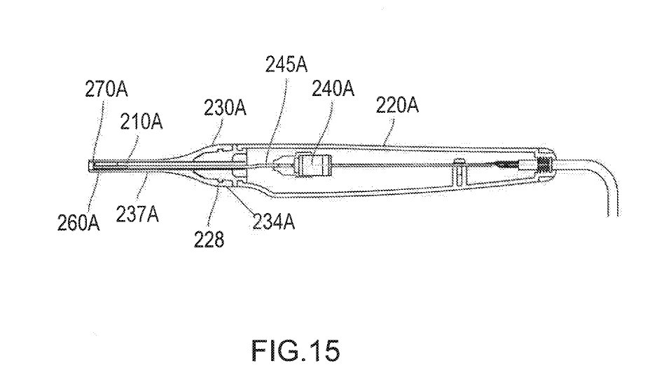

FIG. 15 is a perspective view of the examination tool 200A according to the second embodiment.

The cap part 230A is attached to the leading end part of the main body part 220A. The cap part 230A is firmly attached to the main body part 220A by the locking pieces 228 provided at the leading end part and the engagement grooves 234A in the cap part 230A itself. The imaging tube 210A protruding from the main body part 220A penetrates the protective part 237A of the cap part 230A. The protective part 237A protects the entire circumference of the imaging tube 210A.

A camera 240A that images an inner part of an ear is built in the main body part 220A. An optical fiber 245A is attached to the camera 240A. The optical fiber 245A is connected, through the imaging tube 210A, to an imaging lens 260A provided at the leading end part of the imaging tube 210A. Thus, the camera 240A is connected to the imaging lens 260A via the optical fiber 245A. Also, the imaging lens 260A and an LED 270A, which functions as the illumination unit, are arranged side by side in a radial direction in the leading end part of the imaging tube 210A.

Thus, the imaging lens 260A is prevented, with heat generated by the LED 270A, from being fogged due to humidity inside an ear canal.

### [Operation of Examination Tool]

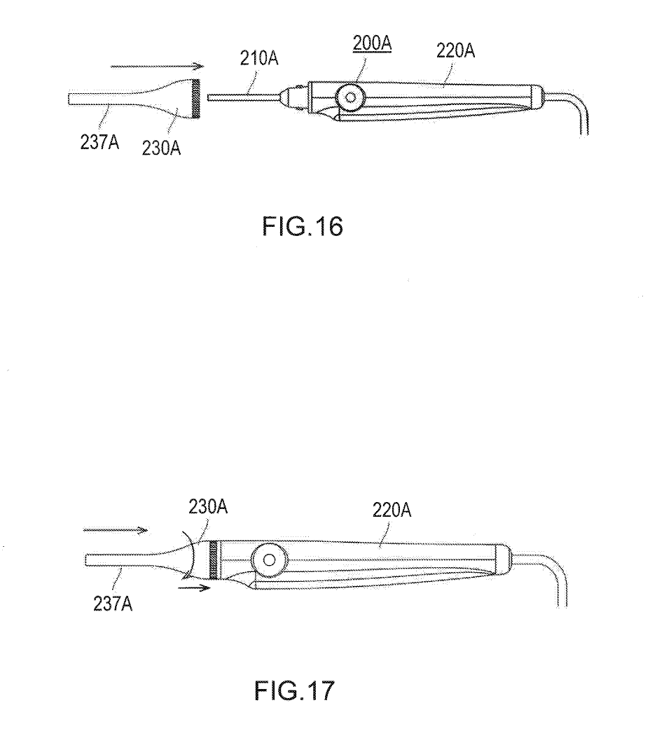

FIGS. 16 to 18 are views for describing the operation of the examination tool 200A according to the second embodiment. As illustrated in FIG. 16, when the examination tool 200A is used, first, the protective part 237A of the cap part 230A is inserted into the imaging tube 210A, and the cap part 230A is slid along the imaging tube 210A in a direction of an illustrated arrow until the cap part 230A is abutted to the leading end part of the main body part 220A.

When the cap part 230A is abutted to the leading end part, the cap part 230A is rotated in the first direction (rightward direction in drawing) as illustrated in FIG. 17. Then, the engagement grooves 234A illustrated in FIG. 14A, 14B are engaged with the locking pieces 228 in FIG. 2, and the cap part 230A is further moved in the direction of the illustrated arrow and is firmly attached to the main body part 220A.

In this state, since the imaging tube 210A is covered and protected by the protective part 237A, the imaging tube 210A is inserted into an ear canal and a state of an inner part of an ear is observed. During the observation of the state of the inner part of the ear, the LED 270A illustrated in FIG. 15 is on and the state of the inner part of the ear is imaged by the camera 240A. Although humidity inside the ear canal is high depending on a kind of an animal, the imaging lens 260A is not fogged since the imaging lens 260A is warmed by the heat of the LED 270A,

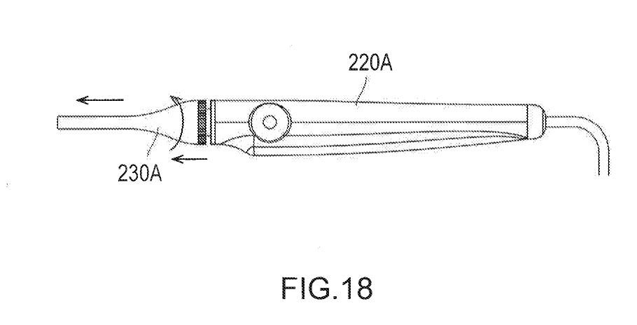

When the observation of the state of the inner part of the ear is over, the cap part 230A is rotated in the second direction (leftward direction in drawing) as illustrated in FIG. 18. Then, the engagement grooves 234A illustrated in FIG. 14A, 14B are detached from the locking pieces 228 in FIG. 2 and the cap part 230A is moved in a direction of an illustrated arrow. Thus, it becomes easier to detach the cap part 230A from the imaging tube 210A,

By an operator, the cap part 230A that becomes easier to be detached is moved in the direction of the illustrated arrow and is detached from the imaging tube 210A illustrated in FIG. 18.

As described above, according to the examination tool of the second embodiment, an introduction cost becomes low since a structure thereof is simple compared to a conventional one. Since fogging of the imaging lens 260A is designed to be prevented, it is possible to observe an inner part of an ear with a clear image. Moreover, since the cap part 230A can be replaced, high reliability can be secured in terms of hygiene.

### Third Embodiment

In the third embodiment, a through hole penetrating the cap portion in the longitudinal direction provided in the cap portion according to the second embodiment.

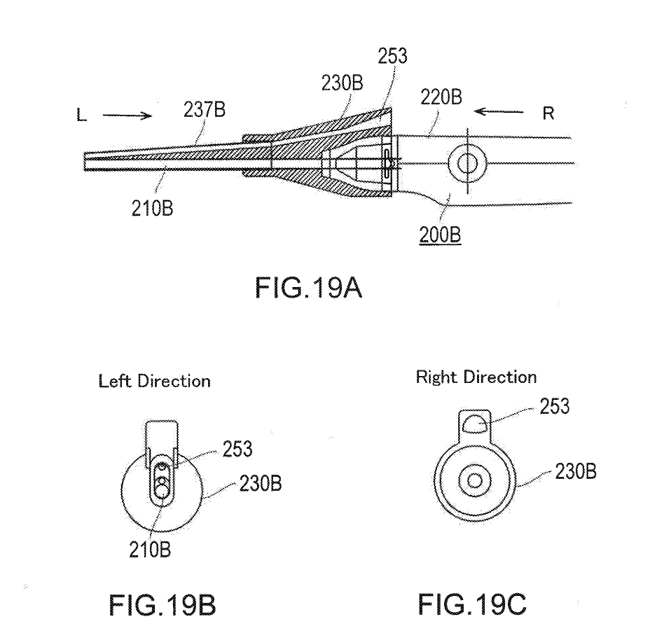

FIG. 19A is a cross section of a cap part of the examination tool according to the third embodiment. FIG. 19B is a Left side view of a cap part of the examination tool according to the third embodiment. FIG. 19C is a Right side view of a cap part of the examination tool according to the third embodiment.

As shown in FIG.19A, 19B, 19C, a through hole 253 is formed so as to pass through the protective portion 237B in the longitudinal direction of the cap portion 230B.

In the through hole 253, for example, a medical instrument such as a forceps is inserted and held so as to be movable along the through hole 253.

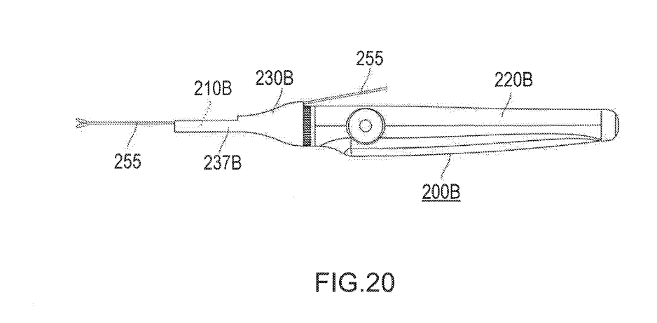

FIG. 20 is a configuration diagram of an examination tool according to third embodiment.

As shown in FIG. 20, the forceps 255 is passed through the through hole 253 shown in FIG. 19. The outer diameter of the forceps 255 is slightly smaller than the inner diameter of the through hole 253. Therefore, the forceps 255 can be moved in the longitudinal direction of the cap portion 230B.

As described above, according to the examination tool of the third embodiment, the examination tool 200B can be provided with a medical device such as forceps 255.

1.-12. (canceled)

13. An examination tool comprising:
an imaging tube that is inserted into an ear canal to image an inner part of an ear; and
a main body part from a leading end part of which the imaging tube protrudes,
wherein the imaging tube includes, at a leading end part thereof, an imaging lens that images the inner part of the ear and an illumination unit that is provided adjacent to the imaging lens and that illuminates the inner part of the ear,
a cap part including a penetration part in which the imaging tube penetrates is included at the leading end part of the main body part,
a protective tube that protects an entire circumference of the imaging tube is attached to the imaging tube,
a holding part that holds an end part of the protective tube with the imaging tube is formed in the penetration part of the cap part, and
the cap part is moved in a direction opposite to a protruding direction of the imaging tube and is fixed to the leading end part when being rotated in a first direction along a circumferential direction of the main body part, and is moved in the protruding direction of the imaging tube, is detached from the leading end part, and moves the protective tube held by the holding part in the protruding direction of the imaging tube when being rotated in a second direction opposite to the first direction.

14. An examination tool comprising:
an imaging tube that is inserted into an ear canal to image an inner part of an ear; and
a main body part from a leading end part of which the imaging tube protrudes,
wherein the imaging tube includes, at a leading end part thereof, an imaging lens that images the inner part of the ear and an illumination unit that is provided adjacent to the imaging lens and that illuminates the inner part of the ear,
a cap part including a protective part in which the imaging tube penetrates and which protects an entire circumference of the imaging tube is included at the leading end part of the main body part, and
the cap part is moved in a direction opposite to a protruding direction of the imaging tube and is fixed to the leading end part when being rotated in a first direction along a circumferential direction of the main body part, and is moved in the protruding direction of the imaging tube and detached from the leading end part when being rotated in a second direction opposite to the first direction.

15. The examination tool according to claim 13, wherein the imaging lens and the illumination unit are arranged side by side in a radial direction in the imaging tube.

16. The examination tool according to claim 13, wherein the imaging lens is prevented by heat generated by the illumination unit from being fogged due to humidity inside the ear canal.

17. The examination tool according to claim 13, wherein the illumination unit is an LED, and a camera is connected to the imaging lens via an optical fiber.

18. The examination tool according to claim 17, wherein the camera is built in the main body part.

19. The examination tool according to claim 13, wherein a locking piece is provided at the leading end part of the main body part,
an engagement groove is provided in the cap part, and
the locking piece is engaged with the engagement groove and the cap part is moved in the direction opposite to the protruding direction of the imaging tube when the cap part is abutted to the leading end part and the cap part is rotated in the first direction, and the cap part is moved in the protruding direction of the imaging tube until the engagement groove is detached from the locking piece when the cap part is rotated in the second direction.

20. The examination tool according to claim 13, wherein the protective tube protects the entire circumference of the imaging tube in a state of protruding more than the leading end part of the imaging tube.

21. The examination tool according to claim 13, wherein the holding part is a protrusion protruding in the protruding direction of the imaging tube from the penetration part of the cap part,
a diameter of the penetration part is equal to a diameter of the imaging tube or larger than the diameter of the imaging tube,
the penetration part includes an abutment part to which the end part of the protective tube is abutted, and
a distance from a surface of the protrusion to a surface of the imaging tube is equal to a thickness of the protective tube or shorter than the thickness of the protective tube.

22. The examination tool according to claim 14, wherein the protective part of the cap part protects the entire circumference of the imaging tube in a state of protruding more than the leading end part of the imaging tube.

23. The examination tool according to claim 14, wherein the protective part is formed integrally with the cap part.

24. The examination tool according to claim 14, wherein the cap part has a through hole penetrating in the longitudinal direction of the cap part for inserting and holding a medical device.

25. The examination tool according to claim 14, wherein the imaging lens and the illumination unit are arranged side by side in a radial direction in the imaging tube.

26. The examination tool according to claim 14, wherein the imaging lens is prevented by heat generated by the illumination unit from being fogged due to humidity inside the ear canal.

27. The examination tool according to claim 14, wherein the illumination unit is an LED, and a camera is connected to the imaging lens via an optical fiber.

28. The examination tool according to claim 14, wherein a locking piece is provided at the leading end part of the main body part,
an engagement groove is provided in the cap part, and
the locking piece is engaged with the engagement groove and the cap part is moved in the direction opposite to the protruding direction of the imaging tube when the cap part is abutted to the leading end part and the cap part is rotated in the first direction, and the cap part is moved in the protruding direction of the imaging tube until the engagement groove is detached from the locking piece when the cap part is rotated in the second direction.
